(19) United States
(12) Patent Application Publication
(10) Pub. No.: US 2009/0170810 A1
(43) Pub. Date: Jul. 2, 2009

(54) METHODS OF TREATMENT OF CARDIOVASCULAR AND CEREBROVASCULAR DISEASES WITH LOW MOLECULAR WEIGHT FUCOIDAN

(75) Inventor: Shouzhu HAO, Beijing (CN)

Correspondence Address: MATTHIAS SCHOLL, 14781 MEMORIAL DRIVE, SUITE 1319, HOUSTON, TX, 77079, US

(73) Assignee: Beijing Century Biocom Pharmaceutical Technology Co. Ltd., Beijing (CN)

(21) Appl. No.: 12/397,428

(22) Filed: Mar. 4, 2009

Related U.S. Application Data
(63) Continuation of application No. PCT/CN2007/002620, filed on Aug. 31, 2007.

(30) Foreign Application Priority Data
Sep. 4, 2006 (CN) 200610127925.8
Dec. 8, 2006 (CN) 200610140395.0

(51) Int. Cl. A61K 31/715; A61P 9/00

(52) U.S. Cl. 514/54

(57) ABSTRACT

A method for treating an ischemic cardiovascular or cerebrovascular disease comprising administrating to a patient in the need of such treatment a pharmaceutical composition comprising low molecular weight fucoidan.

### CROSS-REFERENCE TO RELATED APPLICATIONS

This application is a continuation of International Patent Application No. PCT/CN2007/002620 with an international filing date of Aug. 31, 2007, designating the United States, now pending, and further claims priority benefits to Chinese Patent Application No. 200610127925.8 filed Sep. 4, 2006, and to Chinese Patent Application No. 200610140395.0 filed Dec. 8, 2006. The contents of all of the aforementioned applications, including any intervening amendments thereto, are incorporated herein by reference.

### BACKGROUND OF THE INVENTION

1. Field of the Invention

This invention relates to methods of treating cardiovascular and/or cerebrovascular diseases with low molecular weight fucoidan.

2. Description of the Related Art

Fucoidans are a class of sulfated polysaccharides found mainly in various species of brown seaweed. Fucoidans were first isolated in 1913 from Laminaria digitata (oarweed) by Kylin who initially named them fucoidin because of L-fucose found in the acid hydrolyzate of the seaweed. Subsequently, this class of polysaccharides began to be referred to as fucoidans following standard IUPAC nomenclature. Nevertheless, other names for this class of polysaccharides are also in use including fucan, sulfated fucan, fucosan, fucosan sulfuric ester, fucus polysaccharide, fucose polysaccharide, brown algae syrup, or brown algae polysaccharide sulfuric ester.

The chemical makeup of many fucoidans has since been fully elucidated. Fucoidans have complex chemical structure, mainly comprising fucose and sulfate groups, and additionally often also comprising various groups derived from other compounds, such as galactose, xylose, uronic acid, depending on which algae the fucoidans are isolated from. For example, fucoidan from kelp is composed of different monosaccharides, such as fucose, galactose, xylose, glucuronic acid, arabinose, and so on, and particularly fucose and galactose being present in the weight ratio of about 3:1.

The chemical structure of fucoidans is complex, and varies greatly in different algae. Up to now, the structure of fucoidans extracted from Fucus vesiculosus and Ascophyllum nodosum has been most studied. The fucoidan from Fucus vesiculosus is mainly linked by α(1→3) glycosidic bonds, and the sulfation mainly occurs at the C2 and C3 positions. The fucoidan from Ascophyllum nodosum contains a large number of α(1→3) and α(1→4) glycosidic bonds.

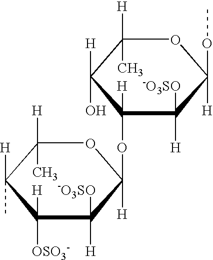

Repeat unit of fucoidan isolated from Fucus vesiculosus and Ascophyllum nodosum

The structure of fucoidan from other brown algae has also reported. For example, the fucoidan from Ecklonia kurome is mainly linked by α(1→3) glycosidic bonds, and sulfation occurs at the C₄ position. The main chain of fucoidan from Cladosiphon okamuranus and Chorda filum comprises fucose linked by α(1→3) glycosidic bonds, and sulfation occurs at the C4 position; furthermore, the fucoidans of the two species comprise a few of 2-O-acetyls groups.

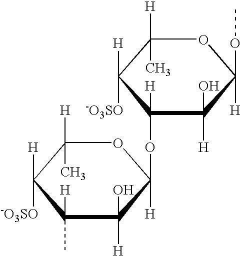

Repeat unit of fucoidan isolated from Ecklonia kurome

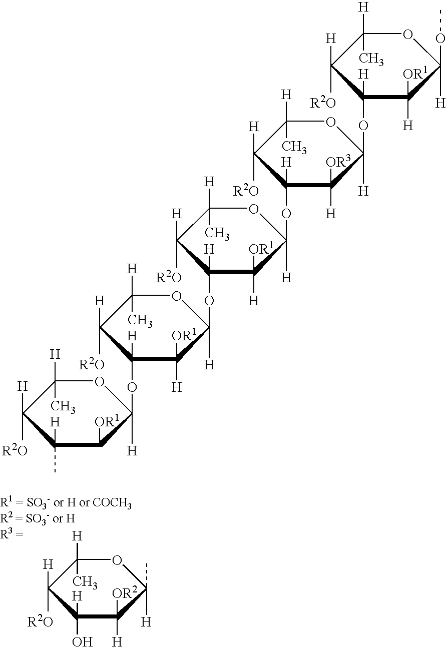

Repeat unit of fucoidan isolated from Chorda filum

It has been shown that the fucoidan from kelp is mainly composed of L-fucose linked by α(1→3) glycosidic bonds, and sulfation occurs at C2 or C4 position. Some contend, however, that there are also side chains in the fucoidan from kelp composed of L-fucose linked by (1→2) glycosidic bonds. This structure would be similar to the structure of fucoidan from Chorda filum shown above with the exception that there are also acetyl groups in Chorda filum, and the percentage of substituted groups is different between the two species. Furthermore, the fucoidan from kelp comprises monosaccharides, such as galactose, xylose, and rhamnose. Galactose may be involved in constituting the main chain, while the xylose and rhamnose may be involved in constituting the side chain.

Preparation methods and medical application of low molecular weight fucoidans have been disclosed in literature. For example, Jap. Patent No. 46-2248 discloses that reacting cetyl pyridine chloride or cetyltrimethylammonium bromide with fucoidan yields a quaternary ammonium salt complex. According to the solubility difference of the complex in salt, algin, a neutral polysaccharide and other impurities are removed by purification with ethyl alcohol, methyl alcohol and ion exchange resin and the purified fucoidan is obtained.

CN1129109A discloses an alkali agglutination separation method comprising soaking air-dried kelp, filtering several times, extracting with alcohol twice, washing with alcohol once, regulating the pH range and so on.

CN1344565A discloses a method comprising pre-treating raw materials, stirring and extracting under a certain temperature, centrifugating, concentrating, precipitating with alcohol, dehydrating with anhydrous alcohol and so on.

CN1517356A discloses a method of preparation of fucoidan oligosaccharide comprising dissolving fucoidan in water, adding hydrogen peroxide, hypochlorous acid or nitrous acid and salt thereof, heating the mixture and ultrafiltering with an ultrafiltration membrane having molecular weight cut-off between 3000 and 5000.

CN1560086A discloses a method of preparation of fucoidan having high content of sulfate, comprising extracting brown alga with hot water or acid water to obtain an extract containing fucoidan, concentrating the extract to the weight percentage of polysaccharide to between 2% and 10%, regulating the pH value to between 5 and 8, adding chitosan solution and stirring, centrifuging or filtering to collect deposit, extracting the deposit 2-4 times with 5-10 times the weight of salt solution, centrifuging or filtering to collect a clear solution; desalting the clear solution by dialyzing or ultrafiltering.

CN1616494A discloses a method of preparation of low molecular weight seaweed sulfated polysaccharides between 4 kDa and 100 kDa comprising adding ascorbic acid and hydrogen peroxide to natural seaweed sulfated polysaccharides, degrading at constant temperature for 0.5-3 h, dialyzing or ultrafiltering, and vacuum concentrating.

Additionally, CN1670028A, CN1392160A and CN1197674A each disclose a flocculation method of preparing algal polysaccharide.

CN1547478A discloses a use of fucoidan in treating adhesion, arthritis and psoriasis.

Furthermore, the above-mentioned references further disclose that fucoidan has one or more of the following properties: anticoagulative, immunity enhancing, anti-tumoral, anti-viral, decreasing blood glucose, radiation-protective, ascite-suppressing, and so on.

Up to now, a use of low molecular weight fucoidan in treating coronary heart disease and stroke has not been disclosed.

### BRIEF SUMMARY OF THE INVENTION

Therefore, it is one objective of the invention to provide a method for the treatment of ischemic cardiovascular and cerebrovascular diseases.

Specifically, in one embodiment of the invention, provided is a method for the treatment of ischemic cardiovascular and cerebrovascular diseases comprising administrating to a patient in need thereof a pharmaceutical composition comprising low molecular weight fucoidan. The ischemic cardiovascular and cerebrovascular diseases include but are not limited to coronary heart disease and stroke. The coronary heart disease includes but is not limited to symptomless coronary heart disease, angina, cardiac infarction, arrhythmia, sudden death. The stroke includes but is not limited to cerebral hemorrhage and cerebral infarction.

In another embodiment of the invention, provided is a pharmaceutical composition comprising low molecular weight fucoidan. The pharmaceutical composition comprises an effective dose of low molecular weight fucoidan and at least one pharmaceutically acceptable excipient.

The mode of administration of the pharmaceutical composition includes but is not limited to intravenous injection, intramuscular injection, hypodermic injection, topical application, oral administration, and rectal administration.

The dosage form of the pharmaceutical composition includes but is not limited to parenteral solution, lyophilized injectable powder, injection microspheres, liposomes, tablets, capsules, water agent, powder, cataplasma, sprayable solution, granular formulation, soft capsules, drop pills, gel, patch, paste, etc. A parenteral solution, lyophilized injectable powder, tablets, and capsules are preferable. Appropriate dosage form is easily prepared by those skilled in the art according to the prior art and common sense.

In certain classes of the embodiment, low molecular weight fucoidan is obtained through degradation of naturally-occurring sulfated polysaccharide or oligosaccharide substances using an appropriate degradation method including but not limited to acid-catalyzed hydrolysis, base-catalyzed hydrolysis, enzymatic depolymerization, mechanical degradation, and oxidative depolymerisation. The molecular weight (in Dalton) is much lower than that of naturally-occurring polysaccharides, and is, e.g., between 8000 and 100000, particularly between 8000 and 60000, more particularly between 8000 and 12000, as well as between 20000 and 40000.

In certain classes of the embodiment, the fucoidan is extracted from kelp, or from wild brown algae such as gulfweed, Undaria pinnatifida, Sargassum fusiform, Sargassum thunbergii, Sargasnam kjellmanianum, Ecklonia kurome, Fucus vesiculosus and Ascophyllum nodosum, etc. In particular, the fucoidan used in the methods of this invention is extracted from kelp.

In certain classes of the embodiment, the weight percentage of low molecular weight fucoidan of the pharmaceutical composition is ≧50%, particularly ≧70%, more particularly ≧90%, and the most particularly ≧95%.

The fucoidan content in a unit-dose is between 1 mg and 1000 mg, particularly between 10 mg and 800 mg, more particularly between 20 mg and 500 mg, or between 20 mg and 300 mg, and the most particularly between 30 mg and 100 mg.

In certain classes of the embodiment, low molecular weight fucoidan can decrease the degree and range of myocardial infarction, and/or reduce the size of myocardial infarction. Particularly, the molecular weight of fucoidan is preferably between 8 kDa and 12 kDa and between 20 kDa and 40 kDa, more particularly between 20 kDa and 40 kDa.

In certain classes of another embodiment, low molecular weight fucoidan decreases ischemia reperfusion-induced brain edema, reduces intracranial pressure, improves brain microcirculation, promotes the generation of superoxide dismutase, and meanwhile reduces the vitality of LDH. Particularly, the molecular weight of fucoidan is particularly between 8 kDa and 12 kDa and between 20 kDa and 40 kDa, and more particularly between 8 kDa and 12 kDa.

The fucoidan of the present invention can be extracted, purified and fractionated according to the following methods:

1. Extracting
Fucoidan was extracted with water, diluted acid or calcium chloride solution, then lead hydroxide, aluminum hydroxide, ethanol, or quaternary ammonium salts cationic surfactants were added to the extract, so that fucoidan precipitated out. In order to reduce the dissolution of pigment and proteins, algae can be pre-treated with a high concentration of alcohol or formaldehyde solution prior to extraction. Techniques such as microwave extraction, ultrasonic extraction, and flocculation polymer precipitation extraction can be used.
2. Purifying
The crude fucoidan obtained from step (1) often contained part of water-soluble alginate, protein, laminaran or pigment and needed to be further purified, the purification methods comprising:
Ethanol Re-Precipitation Method
Crude fucoidan aqueous solution was extracted with hot water and 20% ethanol was added in the presence of 0.05M MgCl₂ to remove impurities such as water-soluble algin (Nishide Eiichi, etc., Bulletin of the Japanese Society of Scientific Fisheries, 1982, 48(12):1771).
Crude fucoidan extracted from Sargassum horneri (turn) was dissolved in water, 4M CaCl₂ and 30% ethanol were added successively to remove algin, then 80% ethanol was added and purified fucoidan precipitated out (Wang Zuoyun, Zhao Xuewu, Isolation and purification of fucoidan, laminaran and algin from Sargassum horneri (turn), Journal of Fisheries of China, 1985, 9(1):71).
Quaternary ammonium salts precipitation method: fucoidan precipitated out by reacting cationic surfactants such as cetyl pyridine chloride (CPC) or cetyltrimethylammonium bromide (CTAB) with a polymer electrolyte.
In the extraction and purification process, a dialysis method is generally used for the removal of ions and small molecules. An ultrafiltration separation method is also used to exclude the smaller molecular weight substances. An enzymatic digestion is sometimes used to remove laminaran and proteins which are intermixed in an extract solution.
Glucanase and alcalase can be used for the removal of laminaran and proteins during extraction and purification process (Fleury N and Lahaye M; Studies on by-products from the industrial extraction of alginate 2. Chemical structure analysis of fucans from the leach-water. J Appl Phycol, 1993, 5: 605-610). Additionally, since laminaran is electrically neutral and fucoidan is generally in the form of polyanions, ion exchange resin method can be used to separate the two compounds.
3. Fractionation
Fucoidan has a complex chemical structure which makes chromatographic and electrophoretic fractionation of crude fucoidan mixtures feasible. A conventional fractionation method involves ethanol precipitation, i.e., a stepwise increasing concentration of ethanol is used to precipitate out different fractions.
Another method involves chromatographic fractionation, e.g., gel filtration chromatography or ion exchange chromatography. Ion-exchange chromatography separates polysaccharides into fractions having different electric charge, and gel filtration chromatography separates polysaccharides according to molecular weight.
Additionally, an ultrafiltration membrane of a certain molecular weight rating can be used to fractionate fucoidans so as to obtain fractions having a certain molecular weight.

Methods of degrading fucoidan to obtain low molecular weight fucoidan include:
1. Acid-catalyzed hydrolysis: glycosidic bonds of polysaccharide are easily broken in acid solution so that the polysaccharide is degraded into low-molecular-weight fragments. Products with various molecular weights can be obtained by regulating acid concentration, reaction temperature and time. It is difficult to control the molecular weight range of polysaccharide fractionation products, and the content of sulfate group varies greatly.
2. Base-catalyzed hydrolysis: the character of acidic polysaccharides changes and the sulfate groups detach easily under alkaline conditions, so the method is not suitable for fucoidan fractionation.
3. Enzymatic degradation: i.e., a specific glycosidase is used to break a certain glycosidic bond of polysaccharide. More and more attention has been paid to enzymatic fractionation methods because these methods possess a high degree of specificity, efficiency, without side effects, and it is easy to control the fractionation conditions and processes. However, due to strong enzyme specificity, these methods are not broadly applicable, as the enzyme is relatively difficult and expensive to manufacture, and easily loses activity.
4. Mechanical degradation: including ultrasonic and microwave degradation. Because of high energy consumption, intensive equipment use, small batch size, mechanical degradation cannot be applied in industrial production. Results of ultrasonication have shown that regardless of the duration of radiation, the fractionated products have the lowest molecular weight limit, and moreover, have a very narrow molecular weight distribution.
5. Oxidative depolymerisation: for example, heparin is degraded by hydrogen peroxide to give products having a high level of sulfation. The method has a low cost and great application value.

In another embodiment of the invention, low molecular weight fucoidan are prepared by following methods: kelp was crushed, soaked in formaldehyde solution overnight, and then distilled water was added. The mixture was boiled to yield an extract. The extracted was filtered with diatomite. The filtrate was firstly dialyzed for a day with running tap water, and then dialyzed for another day with distilled water. The dialysate was concentrated, and ethanol was added dropwise (until the concentration of ethanol was up to 75%) to obtain a precipitate. The precipitate was dried to give a crude fucoidan. The crude product was re-dissolved in water, 20% ethanol was added in the presence of 0.05 mol/L MgCl₂ to precipitate and remove water-soluble algin. The filtrate was dialyzed, concentrated, and precipitated with 75% ethanol, dried to give purified fucoidan. An appropriate amount of fucoidan from kelp was dissolved in distilled water, moderate ascorbic acid and hydrogen peroxide added, mixed and stirred at room temperature. The resultant solution was dialyzed, ultrafiltered, vacuum concentrated, and freeze-dried.

### DETAILED DESCRIPTION OF THE EMBODIMENTS

Detailed description will be given below with reference to accompanying examples. The examples are provided herein to just describe the present invention, it will be obvious to those skilled in the art that changes and modifications may be made without departing from the invention in its broader aspects, and therefore, the aim in the appended claims is to cover all such changes and modifications as fall within the true spirit and scope of the invention.

### Example 1

### Preparation of Fucoidan

Seaweed was crushed, soaked in 3.7% formaldehyde solution overnight, and then distilled water was added. The mixture was boiled to yield an extract. The extracted was filtered with diatomite. The filtrate was firstly dialyzed for a day with running tap water, then dialyzed for another day with distilled water.

The dialysate was concentrated, ethanol added (until the concentration of ethanol was up to 75%), precipitated and dried to give a crude fucoidan. The crude product was re-dissolved in water, 20% ethanol was added in the presence of 0.05 M MgCl₂ to precipitate and remove water-soluble algin. The filtrate was dialyzed, concentrated, precipitated with 75% ethanol, and dried to give a purified fucoidan.

Following the above-mentioned method, fucoidans from four kinds of seaweeds, namely, Sargassum kjellmanianum, Sargassum thunbergii, Sargassum ilicifolium, and kelp were separately prepared.

The chemical composition of the obtained fucoidans is listed below:

| | | | Peak | | | | | |
|---|---|---|---|---|---|---|---|---|
| | | | molecular | | Molar ratio of | | | |
| | Fucose | SO₄²⁻ | weight | Ash | monosaccharide | | | |
| Seaweed | (%) | (%) | (kDa) | (%) | Fucose | Galactose | Xylose | Glucose |
| Sargassum | 26.5 | 14.8 | 980 | 20.8 | 1.00 | 0.24 | 0.05 | 0.04 |
| kjellmanianum | | | | | | | | |
| Sargassum | 25.4 | 17.0 | 650 | 22.6 | 1.00 | 0.24 | | 0.03 |
| thunbergii | | | | | | | | |
| Sargassum | 13.3 | 12.5 | 588 | 20.8 | 1.00 | 0.35 | 0.16 | 0.08 |
| ilicifolium | | | | | | | | |
| kelp | 28.8 | 30.2 | 250 | 31.2 | 1.00 | 0.36 | | |

Preparation of Low Molecular Weight Fucoidan (“Sample A”)

150 g of fucoidan from kelp was dissolved in 10 L of distilled water to give a solution with a (w/v) concentration of 1.5%. Ascorbic acid and hydrogen peroxide were added until the concentration of the two components reached 30 mmol/L, respectively. The solution was mixed until it was homogeneous, and reacted with stirring for 2 hours at room temperature.

After reaction completion, the solution was dialyzed, ultrafiltered, vacuum concentrated, and freeze-dried to give a low molecular weight fucoidan A. The molecular weight was between 8 kDa and 12 kDa. The number average molecular weight was 8.5 kDa, the peak molecular weight was 9.6 kDa, and the weight average molecular weight was 11 kDa. The molecular weight was measured by high-performance gel permeation chromatography (HPGPC). The chemical composition analysis showed: fucose, 28.3%; and sulfate groups, 28.7%.

Preparation of Low Molecular Weight Fucoidan (“Sample B”)

150 g of fucoidan from kelp was dissolved in 10 L of distilled water to give a solution with a concentration (w/v) of 1.5%. Ascorbic acid and hydrogen peroxide were added until the concentration of the two components reached 5 mmol/L, respectively. The solution was mixed until homogeneous, and reacted with stirring for 2 hours at room temperature.

After reaction completion, the solution was dialyzed, ultrafiltered, vacuum concentrated, and freeze-dried to give a low molecular weight fucoidan B. The molecular weight was between 20 kDa and 40 kDa. The number average molecular weight was 25 kDa, the peak molecular weight was 30 kDa, and the weight average molecular weight 34 was kDa. The molecular weight was measured by high-performance gel permeation chromatography (HPGPC). The chemical composition analysis showed: fucose, 28.8%; sulfate groups, 29.1%.

### Example 2

### Preparation of Fucoidan Injection

500 mL of water for injection and 50 g of mannitol were added to 50 g of low molecular weight fucoidan. The pH value being adjusted to 7.0, and the solution was packaged, and freeze-dried.

### Example 3

### Preparation of Low Molecular Weight Fucoidan Tablets

Microcrystalline cellulose and polyvinylpyrrolidone were added to 50 g of low molecular weight fucoidan. After mixing, appropriate amount of water was added, soft materials prepared, granulated, and dried. Crosslinked sodium carboxymethyl cellulose and magnesium stearate were added to the granules, mixed, and tableted. Each tablet has between 10 mg and 200 mg of fucoidan.

### Example 4

### Protection Of Low Molecular Weight Fucoidan Against Myocardial Ischemia

Effect on Hemodynamics and Myocardial Oxygen Consumption in Anesthetized Chest-Open Dogs

Healthy adult dogs (between 12 kg and 20 kg in body mass, male or female) were randomly divided into groups with 6 dogs in each group. The control group was administrated equal volume of 0.9% normal saline, the positive group was administrated a ginkgo biloba extract (4 mg/kg). The experimental group was administrated sample A or sample B. Both sample A and sample B groups had two dosage groups, which were respectively administrated 4 mg/kg, and 16 mg/kg by intravenous injection.

The dogs were anesthetized with i.v. sodium pentobarbital (30 mg/kg), fixed in the back. The neck skin was cut, endotracheal intubation performed to connect an electric respirator. The right carotid artery was exposed, connected to an AP. 601G amplifier, and the blood pressure was measured. The femoral artery was exposed, connected to an AP. 601G amplifier. Ventricular cannulation was performed to measure left ventricular pressure and end diastolic pressure, and ±dp/dt max were measured by a differentiator EQ-601G.

Thoracotomy was performed in the left fourth intercostals, the heart exposed, the pericardium excised, and cardiac surgery performed. The left circumflex coronary artery and aortic root were exposed, and an electromagnetic flowmeter probe was placed to measure coronary blood flow and aortic flow. Limbs were connected to perform limb lead and the standard II lead ECG was measured, and heart rate calculated. Femoral vein was exposed, and venous cannula was performed for drug delivery.

The above-mentioned indexes were simultaneously recorded in a polygraph. After surgery and 15 minutes of stability, indexes were recorded before administration and at 3, 5, 10, 15, 20, 30, 45, 60, 90, 120, 150, 180 and 240 min after administration.

Arterial blood and coronary sinus blood were collected before administration and at 45, 60, 90, 120, 180 and 240 min after administration, blood oxygen content was measured by an oximeter (Kangni-158, US).

The following secondary index was calculated according to formula: mean arterial pressure, cardiac index, stroke index, left ventricular stroke work index, total peripheral resistance, coronary resistance, myocardial oxygen consumption, myocardial oxygen consumption index, myocardial oxygen extraction ratio, myocardial blood flow, and so on. The measured experimental data and percent change were compared with those of the control group, and t-test between groups was performed for statistical analysis.

Effect on Dogs with Experimental Myocardial Infarction

Healthy adult dogs (the same as above) were randomly divided into groups with 6 dogs to each group. The dogs were i.v. anesthetized with pentobarbital sodium (30 mg/kg), fixed in the back. The neck skin was cut, and endotracheal intubation was performed to connect an SC-3 artificial respirator. The lower one third of left anterior descending artery was exposed for ligation to cause myocardial infarction. A wet-type multi-point adsorption method was used to map EECG, provided were 32 mapping points comprising normal area (control points), infarct marginal area and the central area of infarction.

After surgery the dogs were stabilized for 15 minutes. Meanwhile, femoral vein blood was collected and myocardium tris enzyme (AST, CPK, LDH) value was measured as value before administration. After the coronary artery was ligated for 15 minutes, the ST segment was significantly increased, which suggested that a model was established. Through femoral intravenous injection, the control group was administrated equal volume of 0.9% normal saline. The positive group was administrated a ginkgo biloba extract (4 mg/kg). The experimental group was divided into two dose groups, which were respectively administrated 4 mg/kg, and 16 mg/kg of low-molecular weight fucoidan.

EECG was recorded under normal conditions, after ligation, and at 3, 5, 10, 15, 20, 30, 45, 60, 90, 120, 150, 180, 240, 300, 360 min after administration. Σ-ST was expressed as the total increased mV number of the ST-segment, and N-ST was expressed as increased ST-segment lead number >2 mV. At 360 min after administration, blood was collected again to measure myocardium tris enzyme.

After experiment, the heart was harvested and the total weight measured. The root of great vessel and atrial were cut along coronary sulcus to obtain the weight of left ventricle. The left ventricle was cut into 5 or 6 pieces cross-sectionally and equably. The pieces were colored in nitro blue tetrazolium (N-BT) for 15 min in constant temperature water bath at 37° C. The infarcted area was not colored, while the non-infarcted area was colored blue by NBT. The non-infarcted cardiac muscle which had been colored was cut, and the infarcted cardiac muscle which had not been colored was weighted. The weight was divided by the total heart weight and the ventricular weight respectively to obtain the percentage of the infarcted area in the total heart weight and in the ventricular weight.

All experimental data was expressed as X̅±S, and t test was used to determine the significance of difference of mean value between groups.

Results

For sample A, in the dosage group of 16 mg/kg, the measured value of the effect of fucoidan on the degree of ischemia in dogs between 10 min and 150 min after administration was significantly different from that of the control group, and the change rate exhibited a significant inhibitory effect. However, the other dosage group of 4 mg/kg didn't exhibit a significant effect. The results showed that a large amount of sample A can alleviate the degree of ischemia in dogs.

For sample B, in the dosage group of 16 mg/kg, the measured value of the effect of fucoidan on the degree of ischemia in dogs between 3 min and 240 min after administration was significantly different from that of the control group, and the corresponding change rate exhibits a significant inhibitory effect. In the other dosage group of 4 mg/kg, the measured value of the effect of fucoidan on the degree of ischemia in dogs between 60 min and 240 min after administration was significantly different from that of the control group, and the corresponding change rate exhibits a significant inhibitory effect between 60 min and 150 min after administration. The results showed sample B can alleviate the degree of ischemia in dogs.

For sample A, in the dosage group of 16 mg/kg, the measured value of the effect of fucoidan on the range of ischemia in dogs between 15 min and 180 min after administration was significantly different from that of the control group, and the change rate exhibits a significant inhibitory effect between 30 min and 150 min.

For sample B, in the dosage group of 16 mg/kg, the measured value of the effect of fucoidan on the range of ischemia in dogs between 3 min and 240 min after administration was significantly different from that of the control group, and the change rate exhibits a significant inhibitory effect between 30 min and 180 min. In the other dosage group of 4 mg/kg, the measured value of the effect of fucoidan on the range of ischemia in dogs between 60 min and 240 min after administration was significantly different from that of the control group, and the corresponding change rate exhibits a significant inhibitory effect between 120 min and 180 min after administration.

From the above-mentioned results, low molecular weight fucoidan from kelp can decrease the degree and range of myocardial infarction, and reduce the size of myocardial infarction.

The detailed results are listed in Tables 1, 2, 3, 4, 5 and 6.

TABLE 1
Effect of low molecular weight fucoidan (sample A) on the degree of
ischemia in dogs with myocardial infarction (Σ − ST, Mv) (X ± s, n = 6)

| | |
|---|---|
| Dosage | After administration (min) |

| Groups | (mg/kg) | Ligation | 3 | 5 | 10 |
|---|---|---|---|---|---|
| Control | — | 186.00 ± 63.60 | 182.50 ± 47.62 | 167.33 ± 40.71 | 186.50 ± 45.72 |
| group | | | | | |
| % | | | 6.44 ± 43.27 | −2.43 ± 36.90 | 5.03 ± 25.18 |
| Ginkgo | 4.0 | 195.83 ± 55.89 | 189.83 ± 92.59 | 182.17 ± 78.42 | 146.33 ± 48.13 |
| Biloba | | | | | |
| extract | | | | | |
| % | | | −6.98 ± 20.97 | −8.66 ± 30.77 | −24.36 ± 22.46 |
| Sample A | 4.0 | 165.33 ± 68.23 | 153.17 ± 49.63 | 140.83 ± 27.64 | 138.00 ± 28.24 |
| % | | | −3.24 ± 19.26 | −5.93 ± 34.15 | −8.76 ± 31.04 |
| Sample A | 16.0 | 141.33 ± 51.92 | 130.50 ± 53.75 | 135.00 ± 63.20 | 126.00 ± 39.13* |
| % | | | −6.18 ± 25.36 | −2.92 ± 25.60 | −6.03 ± 23.88 |

| | After administration (min) | | | | | |
|---|---|---|---|---|---|---|
| | 15 | 20 | 30 | 45 | 60 | 90 |
| Control | 180.17 ± 58.45 | 173.67 ± 51.72 | 172.50 ± 52.51 | 160.67 ± 35.97 | 175.00 ± 55.68 | 171.67 ± 75.79 |
| group | | | | | | |
| % | 1.14 ± 31.98 | −0.82 ± 36.50 | −1.02 ± 34.45 | −7.21 ± 29.09 | 2.40 ± 48.24 | −0.90 ± 46.91 |
| Ginkgo | 115.67 ± 23.31* | 113.17 ± 15.74* | 97.50 ± 23.17** | 85.00 ± 15.88*** | 89.67 ± 29.06** | 90.67 ± 23.24* |
| Biloba | | | | | | |
| extract | | | | | | |
| % | −36.33 ± 20.69# | −39.56 ± 12.95# | −47.91 ± 14.66# | −54.15 ± 12.07## | −53.33 ± 11.82# | −50.98 ± 15.48# |
| Sample A | 134.67 ± 41.36 | 140.83 ± 51.25 | 146.17 ± 42.80 | 144.50 ± 37.55 | 140.33 ± 44.68 | 130.83 ± 34.83 |
| % | −13.47 ± 27.17 | −11.29 ± 24.66 | −5.14 ± 30.90 | −6.34 ± 26.25 | −10.32 ± 24.11 | −15.78 ± 21.36 |
| Sample A | 111.50 ± 45.29* | 96.83 ± 17.99** | 93.67 ± 12.57** | 87.83 ± 25.80** | 93.33 ± 33.40* | 94.33 ± 28.83* |
| % | −14.86 ± 32.15 | −24.58 ± 24.57 | −24.38 ± 31.11 | −29.93 ± 32.17 | −29.67 ± 25.43 | −28.75 ± 25.72 |

| | After administration (min) | | | | | |
|---|---|---|---|---|---|---|
| | 120 | 150 | 180 | 240 | 300 | 360 |
| Control | 170.83 ± 60.97 | 163.50 ± 49.94 | 160.67 ± 48.39 | 171.33 ± 70.57 | 166.17 ± 64.44 | 164.83 ± 57.85 |
| group | | | | | | |
| % | −1.75 ± 41.95 | −5.29 ± 39.29 | −5.46 ± 42.57 | −0.37 ± 47.76 | −1.24 ± 49.80 | −3.96 ± 40.74 |
| Ginkgo | 78.50 ± 17.06** | 79.33 ± 20.61** | 94.00 ± 34.05* | 97.17 ± 36.90* | 100.33 ± 34.48 | 103.33 ± 37.66 |
| Biloba | | | | | | |
| extract | | | | | | |
| % | −56.13 ± 19.53# | −54.67 ± 23.30# | −45.77 ± 32.33 | −43.64 ± 36.39 | −44.08 ± 31.33 | −41.78 ± 35.48 |
| Sample A | 127.17 ± 33.08 | 135.50 ± 43.99 | 138.83 ± 59.56 | 129.67 ± 59.08 | 139.67 ± 54.10 | 145.83 ± 45.76 |
| % | −16.38 ± 30.84 | −6.34 ± 57.80 | −7.23 ± 58.63 | −9.79 ± 69.49 | −1.44 ± 71.40 | 2.17 ± 67.27 |
| Sample A | 90.17 ± 39.02* | 100.67 ± 44.11* | 104.67 ± 44.20 | 116.33 ± 55.39 | 104.50 ± 40.41 | 103.83 ± 35.29 |
| % | −30.82 ± 34.60 | −22.57 ± 42.81 | −20.51 ± 40.07 | −9.07 ± 55.87 | −22.37 ± 32.99 | −24.33 ± 22.68 |

TABLE 2
Effect of low molecular weight fucoidan (sample A) on the range of
ischemia in dogs with myocardial infarction (N-ST, points) (X ± s, n = 6)

| | |
|---|---|
| Dosage | After administration (min) |

| Groups | (kg/mg) | Ligation | 3 | 5 | 10 |
|---|---|---|---|---|---|
| Control | — | 21.50 ± 2.95 | 21.50 ± 3.21 | 20.83 ± 3.25 | 20.17 ± 3.13 |
| group | | | | | |
| % | | | −0.31 ± 3.27 | −3.24 ± 5.10 | −5.89 ± 9.70 |
| Ginkgo | 4.0 | 19.50 ± 2.17 | 19.50 ± 2.07 | 16.50 ± 4.14 | 16.50 ± 2.88 |
| Biloba | | | | | |
| extract | | | | | |
| % | | | 0.55 ± 11.97 | −15.77 ± 17.84 | −14.44 ± 18.47 |
| Sample A | 4.0 | 20.17 ± 4.12 | 19.00 ± 4.56 | 18.17 ± 5.31 | 17.83 ± 5.60 |
| % | | | −5.61 ± 12.68 | −10.77 ± 12.33 | −12.49 ± 15.84 |
| Sample A | 16.0 | 19.67 ± 2.34 | 18.00 ± 2.37 | 17.33 ± 3.20 | 17.50 ± 2.51 |
| % | | | −7.81 ± 13.19 | −11.01 ± 17.78 | −10.32 ± 14.33 |

| | After administration (min) | | | | | |
|---|---|---|---|---|---|---|
| | 15 | 20 | 30 | 45 | 60 | 90 |
| Control | 20.17 ± 2.86 | 19.83 ± 2.64 | 20.83 ± 2.64 | 19.83 ± 3.97 | 21.33 ± 2.25 | 20.83 ± 1.94 |
| group | | | | | | |
| % | −5.59 ± 12.07 | −7.47 ± 6.52 | −2.91 ± 4.62 | −8.13 ± 11.17 | 0.33 ± 14.61 | −1.39 ± 18.38 |
| Ginkgo | 16.67 ± 3.27 | 15.83 ± 2.48* | 15.17 ± 2.71** | 12.83 ± 3.55** | 12.83 ± 3.60*** | 12.67 ± 3.83*** |
| Biloba | | | | | | |
| extract | | | | | | |
| % | −13.39 ± 22.25 | −17.63 ± 18.48 | −21.21 ± 16.75# | −34.13 ± 17.12# | −33.53 ± 19.03## | −35.08 ± 18.85# |
| Sample A | 17.33 ± 6.74 | 17.88 ± 5.08 | 19.83 ± 3.43 | 20.00 ± 3.58 | 18.33 ± 4.27 | 18.00 ± 3.63 |
| % | −15.46 ± 23.69 | −11.66 ± 17.24 | −0.10 ± 15.81 | 1.20 ± 20.17 | −7.13 ± 25.01 | −8.29 ± 24.46 |
| Sample A | 15.33 ± 4.46* | 13.67 ± 3.45** | 13.50 ± 4.64** | 13.50 ± 4.59* | 12.67 ± 4.27** | 14.17 ± 4.36** |
| % | −21.20 ± 25.03 | −29.08 ± 22.89 | −30.88 ± 25.01# | −30.55 ± 26.40 | −34.04 ± 26.85# | −27.60 ± 24.07 |
| | After administration (min) | | | | | |
| | 120 | 150 | 180 | 240 | 300 | 360 |
| Control | 20.00 ± 2.90 | 19.83 ± 2.04 | 19.83 ± 2.79 | 19.33 ± 2.25 | 17.83 ± 2.48 | 18.17 ± 3.19 |
| group | | | | | | |
| % | −5.22 ± 21.49 | −6.71 ± 13.25 | −6.71 ± 15.43 | −8.83 ± 14.59 | −15.51 ± 17.04 | −14.35 ± 16.87 |
| Ginkgo | 13.33 ± 3.72** | 13.00 ± 2.90*** | 14.50 ± 2.35** | 15.00 ± 3.10* | 14.33 ± 3.08 | 14.00 ± 4.52 |
| Biloba | | | | | | |
| extract | | | | | | |
| % | −32.12 ± 15.12# | −33.62 ± 10.78## | −25.79 ± 6.94# | −22.89 ± 15.13 | −25.76 ± 19.42 | −26.96 ± 28.35 |
| Sample A | 18.00 ± 4.65 | 19.17 ± 5.12 | 18.33 ± 5.75 | 18.17 ± 4.71 | 17.50 ± 5.54 | 17.67 ± 4.08 |
| % | −8.59 ± 27.04 | −2.87 ± 29.88 | −7.37 ± 31.98 | −8.03 ± 27.51 | −12.46 ± 26.39 | −11.89 ± 13.51 |
| Sample A | 13.00 ± 3.58** | 13.50 ± 3.15** | 14.33 ± 3.72* | 15.50 ± 4.93 | 15.50 ± 4.46 | 14.00 ± 16.87 |
| % | −33.07 ± 22.04 | −30.64 ± 19.44# | −26.54 ± 20.71 | −21.21 ± 22.55 | −21.06 ± 20.37 | −28.68 ± 19.45 |

TABLE 3
Effect of low molecular weight fucoidan (sample B) on the degree of
ischemia in dogs with myocardial infarction (N-ST, points) (X ± s, n = 6)

| | |
|---|---|
| Dosage | After administration (min) |

| Groups | (kg/mg) | Ligation | 3 | 5 | 10 |
|---|---|---|---|---|---|
| Control | — | 186.00 ± 63.60 | 182.50 ± 47.62 | 167.33 ± 40.71 | 186.50 ± 45.72 |
| group | | | | | |
| % | | | 6.44 ± 43.27 | −2.43 ± 36.90 | 5.03 ± 25.18 |
| Ginkgo | 4.0 | 195.83 ± 55.89 | 189.83 ± 92.59 | 182.17 ± 78.42 | 146.33 ± 48.13 |
| Biloba | | | | | |
| extract | | | | | |
| % | | | −6.98 ± 20.97 | −8.66 ± 30.77 | −24.36 ± 22.46 |
| Sample B | 4.0 | 191.67 ± 37.53 | 197.50 ± 59.10 | 176.67 ± 49.36 | 170.50 ± 48.72 |
| % | | | 1.57 ± 14.42 | −7.52 ± 19.68 | −12.09 ± 10.98 |
| Sample B | 16.0 | 140.17 ± 51.78 | 116.00 ± 45.54* | 104.50 ± 48.56* | 117.17 ± 31.73* |
| % | | | −15.47 ± 19.72 | −22.67 ± 26.70 | −13.45 ± 11.00 |

| | After administration (min) | | | | | |
|---|---|---|---|---|---|---|
| | 15 | 20 | 30 | 45 | 60 | 90 |
| Control | 180.17 ± 58.45 | 173.67 ± 51.72 | 172.50 ± 52.51 | 160.67 ± 35.97 | 175.00 ± 55.68 | 71.67 ± 75.79 |
| group | | | | | | |
| % | 1.14 ± 31.98 | −0.82 ± 36.50 | −1.02 ± 34.45 | −7.21 ± 29.09 | 2.40 ± 48.24 | −0.90 ± 46.91 |
| Ginkgo | 115.67 ± 23.31* | 113.17 ± 15.74* | 97.50 ± 23.17** | 85.00 ± 15.88*** | 89.67 ± 29.06** | 90.67 ± 23.24* |
| Biloba | | | | | | |
| extract | | | | | | |
| % | −36.33 ± 20.69# | −39.56 ± 12.95# | −47.91 ± 14.66# | −54.15 ± 12.07## | −53.33 ± 11.82# | −50.98 ± 15.48# |
| Sample B | 164.67 ± 42.46 | 168.83 ± 42.92 | 147.33 ± 36.94 | 131.00 ± 39.75 | 107.50 ± 36.49* | 95.50 ± 38.95 |
| % | −13.12 ± 21.30 | −11.07 ± 20.43 | −22.62 ± 14.75 | −31.32 ± 16.45 | −44.01 ± 15.97# | −50.63 ± 15.52# |
| Sample B | 109.17 ± 34.42* | 115.83 ± 35.01* | 92.83 ± 28.72** | 89.33 ± 24.08** | 84.83 ± 21.66** | 84.33 ± 29.64* |
| % | −17.55 ± 31.24 | −12.84 ± 29.79 | −29.21 ± 29.34 | −32.73 ± 18.99 | −34.67 ± 25.19 | −36.73 ± 23.45 |
| | After administration (min) | | | | | |
| | 120 | 150 | 180 | 240 | 300 | 360 |
| Control | 170.83 ± 60.97 | 163.50 ± 49.94 | 160.67 ± 48.39 | 171.33 ± 70.57 | 166.17 ± 64.44 | 164.83 ± 57.85 |
| group | | | | | | |
| % | −1.75 ± 41.95 | −5.29 ± 39.29 | −5.46 ± 42.57 | −0.37 ± 47.76 | −1.24 ± 49.80 | −3.96 ± 40.74 |
| Ginkgo | 78.50 ± 17.06** | 79.33 ± 20.61** | 94.00 ± 34.05* | 97.17 ± 36.90* | 100.33 ± 34.48 | 103.33 ± 37.66 |
| Biloba | | | | | | |
| extract | | | | | | |
| % | −56.13 ± 19.53# | −54.67 ± 23.30# | −45.77 ± 32.33 | −43.64 ± 36.39 | −44.08 ± 31.33 | −41.78 ± 35.48 |
| Sample B | 98.50 ± 38.26* | 97.50 ± 36.96* | 88.33 ± 47.82* | 90.17 ± 49.83* | 107.17 ± 44.58 | 106.67 ± 41.40 |
| % | −49.15 ± 13.88# | −48.63 ± 18.91# | −51.46 ± 32.26 | −51.63 ± 30.57 | −42.11 ± 30.55 | −42.12 ± 29.37 |
| Sample B | 82.17 ± 31.33* | 88.17 ± 33.11* | 91.50 ± 44.63* | 93.33 ± 45.35* | 99.17 ± 33.14 | 109.33 ± 37.80 |
| % | −40.71 ± 13.93 | −36.25 ± 14.10 | −35.92 ± 19.17 | −33.93 ± 15.55 | −27.21 ± 14.88 | −20.50 ± 18.11 |

Note:
Compared with the normal salt group,
*p < 0.05
**p < 0.01
***p < 0.001;
compared with the change rate of the normal salt group,
#p < 0.05,
##p < 0.01.

TABLE 4
Effect of low molecular weight fucoidan (sample B) on the degree of
ischemia in dogs with myocardial infarction (N-ST, points) (X ± s, n = 6)

| | |
|---|---|
| Dosage | After administration (min) |

| Groups | (kg/mg) | Ligation | 3 | 5 | 10 |
|---|---|---|---|---|---|
| Control | — | 21.50 ± 2.95 | 21.50 ± 3.21 | 20.83 ± 3.25 | 20.17 ± 3.13 |
| group | | | | | |
| % | | | −0.31 ± 3.27 | −3.24 ± 5.10 | −5.89 ± 9.70 |
| Ginkgo | 4.0 | 19.50 ± 2.17 | 19.50 ± 2.07 | 16.50 ± 4.14 | 16.50 ± 2.88 |
| Biloba | | | | | |
| extract | | | | | |
| % | | | 0.55 ± 11.97 | −15.77 ± 17.84 | −14.44 ± 18.47 |
| Sample B | 4.0 | 19.33 ± 2.88 | 18.00 ± 4.00 | 17.83 ± 5.04 | 17.67 ± 5.5 7 |
| % | | | −7.69 ± 9.52 | −9.18 ± 14.34 | −10.07 ± 19.92 |
| Sample B | 16.0 | 18.67 ± 1.51 | 17.17 ± 2.64* | 14.17 ± 5.15* | 15.67 ± 3.72* |
| % | | | −8.35 ± 8.51 | −24.18 ± 25.19 | −15.90 ± 18.41 |

| | After administration (min) | | | | | |
|---|---|---|---|---|---|---|
| | 15 | 20 | 30 | 45 | 60 | 90 |
| Control | 20.17 ± 2.86 | 19.83 ± 2.64 | 20.83 ± 2.64 | 19.83 ± 3.97 | 21.33 ± 2.25 | 20.83 ± 1.94 |
| group | | | | | | |
| % | −5.59 ± 12.07 | −7.47 ± 6.52 | −2.91 ± 4.62 | −8.13 ± 11.17 | 0.33 ± 14.61 | −1.39 ± 18.38 |
| Ginkgo | 16.67 ± 3.27 | 15.83 ± 2.48* | 15.17 ± 2.71** | 12.83 ± 3.55** | 12.83 ± 3.60*** | 12.67 ± 3.83*** |
| Biloba | | | | | | |
| extract | | | | | | |
| % | −13.39 ± 22.25 | −17.63 ± 18.48 | −21.21 ± 16.75# | −34.13 ± 17.12# | −33.53 ± 19.03## | −35.08 ± 18.85# |
| Sample B | 18.83 ± 3.97 | 17.50 ± 5.05 | 16.50 ± 5.32 | 15.67 ± 3.05 | 15.33 ± 5.32* | 14.83 ± 5.19* |
| % | −3.31 ± 8.64 | −10.88 ± 15.40 | −16.03 ± 18.79 | −19.80 ± 19.93 | −21.47 ± 21.78 | −23.86 ± 21.82 |
| Sample B | 15.50 ± 12.07 | 15.83 ± 2.64* | 14.67 ± 3.56** | 14.00 ± 2.68* | 12.83 ± 4.12** | 12.50 ± 4.09** |
| % | −16.83 ± 18.60 | −14.84 ± 14.36 | −21.24 ± 19.34# | −24.55 ± 16.28 | −31.15 ± 21.48# | −32.92 ± 21.67# |

| | After administration (min) | | | | | |
|---|---|---|---|---|---|---|
| | 120 | 150 | 180 | 240 | 300 | 360 |
| Control | 20.00 ± 2.90 | 19.83 ± 2.04 | 19.83 ± 2.79 | 19.33 ± 2.25 | 17.83 ± 2.48 | 18.17 ± 3.19 |
| group | | | | | | |
| % | −5.22 ± 21.49 | −6.71 ± 13.25 | −6.71 ± 15.43 | −8.83 ± 14.59 | −15.51 ± 17.04 | −14.35 ± 16.87 |
| Ginkgo | 13.33 ± 3.72** | 13.00 ± 2.90*** | 14.50 ± 2.35** | 15.00 ± 3.10* | 14.33 ± 3.08 | 14.00 ± 4.52 |
| Biloba | | | | | | |
| extract | | | | | | |
| % | −32.12 ± 15.12# | −33.62 ± 10.78## | −25.79 ± 6.94# | −22.89 ± 15.13 | −25.76 ± 19.42 | −26.96 ± 28.35 |
| Sample B | 13.00 ± 5.02* | 12.83 ± 5.53* | 12.17 ± 5.19** | 12.67 ± 5.96* | 13.00 ± 5.48 | 13.33 ± 4.37 |
| % | −33.90 ± 20.10# | −34.59 ± 23.44# | −37.89 ± 21.91# | −35.63 ± 26.17 | −34.11 ± 22.90 | −32.46 ± 15.82 |
| Sample B | 12.33 ± 2.73*** | 11.00 ± 4.15*** | 11.83 ± 5.42** | 12.67 ± 5.89* | 13.83 ± 3.76 | 12.83 ± 5.38 |
| % | −33.85 ± 14.20# | −40.98 ± 21.92## | −36.84 ± 27.06# | −32.74 ± 28.11 | −25.97 ± 18.49 | −31.30 ± 27.79 |

TABLE 5
Effect of low molecular weight fucoidan A on the myocardial infarct
size in dogs with myocardial infarction (X ± s, n = 6)

| | | | Infarct/Left |
|---|---|---|---|
| Groups | Dosage | Infarct/Heart (%) | ventricular (%) |
| Normal salt | — | 14.75 ± 1.73 | 21.67 ± 2.42 |
| Ginkgo biloba extract | 4.0 mg/kg | 11.74 ± 1.66* | 17.43 ± 2.28* |
| Sample A | 4.0 mg/kg | 12.63 ± 1.06* | 18.30 ± 1.61* |
| Sample A | 16.0 mg/kg | 11.12 ± 1.85** | 16.66 ± 2.70** |

TABLE 6
Effect of low molecular weight fucoidan B on the myocardial infarct
size in dogs with myocardial infarction (X ± s, n = 6)

| | | | Infarct/Left |
|---|---|---|---|
| Groups | Dosage | Infarct/Heart (%) | ventricular (%) |
| Normal salt | — | 14.75 ± 1.73 | 21.67 ± 2.42 |
| Ginkgo biloba | 4.0 mg/kg | 11.74 ± 1.66* | 17.43 ± 2.28* |
| extract | | | |
| Sample B | 4.0 mg/kg | 11.29 ± 0.83** | 17.26 ± 1.49** |
| Sample B | 16.0 mg/kg | 9.32 ± 0.41*** | 15.33 ± 1.08*** |

### Example 5

### Protection of Low Molecular Weight Fucoidan Against Experimental Cerebral Ischemia

Method

Effect on Breathing Time, Breathing Frequency and Brain Water Content in Decapitated Mice

ICR mice (equally divided between male and female) were divided randomly in a blank control group, a positive control group and dosage groups of sample A (200, 100, and 50 mg/kg) and sample B (400, 200, 100, 50 mg/kg). The mice in medical groups were administrated by tail intravenous injection, and the dosage was 10 ml/mg. The positive control group was administrated nimodipine (2 mg/kg) by tail intravenous injection. The model group was administrated normal salt. At 15 minutes after administration, the mice were decapitated by a pair of scissors. The mouth breathing time, breathing frequency and brain water content were recorded and compared with other groups.

Measurement of brain water content: whole brains were collected, after the wet weight was obtained, they were dried in an oven at 100° C. for 24 hours, the average value was taken to calculate brain water content: brain water content(%)=(wet weight−dry weight)×100%. Brain index: brain index=brain wet weight (g)/body weight (g)×100%.

Effect on Cerebral Ischemia in Mice with the Common Carotid Artery Ligation and Reperfusion

Experimental grouping: a control group and a model group (respectively administrating an equal volume of normal salt), a positive control group (nimodipine, 2 mg/kg), sample A groups (200, 100, and 50 mg/kg), and sample B groups (200, 100, and 50 mg/kg), the injection dosage being 10 mL/mg.

Animal model setup: the grouped mice were respectively administrated test substance, nimodipine or normal salt by tail intravenous injection. After 15 minutes, the mice were anesthetized with 3.5% chloral hydrate, fixed in the back. The right and left common carotid artery and vagus nerve were exposed, and 4-0 suture was inserted under the bilateral carotid arteries. The line was tightened to block blood flow for 5 minutes. Then the line was loosened to make the blood reperfusion for 10 minutes. The operation was repeated three times, and an ischemia-reperfusion model in mice was established. After the last reperfusion, the mice were decapitated and brain collected. In the control group, only the bilateral carotid arteries were exposed, without line being inserted.

Results

Effect on Breathing Time, Breathing Frequency, Brain Index and Brain Water Content in Decapitated Mice

Effect on breathing time and breathing frequency: for sample A, compared with the blank control group, the dosage group of 200 mg/kg can significantly prolong the breathing time (p<0.01), and can significantly increase the breathing frequency (p<0.01). For sample B, the dosage group of 100 mg/kg can significantly prolong the breathing time, and the dosage group of 400, 100, 50 mg/kg can significantly increase the breathing frequency (p<0.05). The results are shown in Table 7.

TABLE 7
Effect of low molecular weight fucoidan on breathing time and
breathing frequency in decapitated mice

| | | | | Breathing |
|---|---|---|---|---|
| | Dosage | Number of | Breathing | frequency |
| Groups | (mg/kg) | animal | time (s) | (times) |
| Blank control | — | 10 | 15.9 ± 2.6 | 11.9 ± 3.1 |
| Nimodipine | 2 | 10 | 23.8 ± 3.2** | 17.0 ± 3.2** |
| Sample A | 200 | 10 | 22.9 ± 2.5** | 16.8 ± 3.1* |
| Sample A | 100 | 10 | 18.7 ± 2.8* | 13.9 ± 1.5* |
| Sample A | 50 | 10 | 14.9 ± 3.2 | 12.6 ± 2.4 |
| Sample B | 400 | 10 | 17.4 ± 1.8 | 14.8 ± 2.1* |
| Sample B | 200 | 10 | 17.5 ± 1.3 | 12.7 ± 2.2 |
| Sample B | 100 | 10 | 18.1 ± 2.0* | 14.6 ± 1.7* |
| Sample B | 50 | 10 | 17.3 ± 3.0 | 15.3 ± 3.2* |

Effect on brain index and brain water content: for sample A, compared with the blank control group, in the dosage group of 200 mg/kg and 100 mg/kg there was a significant decrease of the brain index and brain water content (p<0.05 or p<0.01); for sample B, in the dosage group of 400 mg/kg there was a significant decrease of the brain water content (p<0.05), which suggests that sample A and B can alleviate brain edema after ischemia-reperfusion, reduce intracranial pressure, and improve brain microcirculation. The results are shown in Table 8.

TABLE 8
Effect of low molecular weight fucoidan on brain water
content in decapitated mice

| | Dosage | Animal | Brain index | Brain water |
|---|---|---|---|---|
| Groups | (mg/kg) | numbers | (%) | content (%) |
| Blank control | — | 10 | 1.59 ± 0.12 | 80.6 ± 2.4 |
| Nimodipine | 2 | 10 | 1.40 ± 0.14** | 75.1 ± 0.9** |
| Sample A | 200 | 10 | 1.39 ± 0.10** | 75.4 ± 1.2** |
| Sample A | 100 | 10 | 1.48 ± 0.07* | 78.5 ± 1.6* |
| Sample A | 50 | 10 | 1.54 ± 0.15 | 81.3 ± 1.8 |
| Sample B | 400 | 10 | 1.48 ± 0.16* | 78.5 ± 1.6* |
| Sample B | 200 | 10 | 1.54 ± 0.13 | 80.1 ± 2.7 |
| Sample B | 100 | 10 | 1.59 ± 0.11 | 80.1 ± 1.7 |
| Sample B | 50 | 10 | 1.57 ± 0.12 | 79.9 ± 1.6 |

Effect on cerebral ischemia in mice with the common carotid artery ligation and reperfusion.

In this embodiment, the LDH level in the model group has significantly increased compared with the control group, the superoxide dismutase level decreased significantly (p<0.01), which suggests the ischemic symptoms of the brain cells death have emerged, nimodipine can promote the generation of superoxide dismutase, meanwhile to lower the vitality of LDH. For sample A, the dosage group of 200 mg/kg and 100 mg/kg can promote the generation of superoxide dismutase, meanwhile to lower the vitality of LDH (p<0.05 or p<0.01). For sample B, the dosage group of 200 mg/kg can significantly decrease the vitality of LDH and promote the generation of superoxide dismutase. The results are shown in Table 9.

TABLE 9
Effect of low molecular weight fucoidan on LDH and superoxide
dismutase content in the brain of ischemic mice

| | Dosage | Animal | | |
|---|---|---|---|---|
| Groups | (mg/kg) | numbers | LDH (U/mg) | SOD (U/mg) |
| Blank control | — | 10 | 10.3 ± 1.7 | 163.87 ± 13.08 |
| Model control | — | 10 | 32.1 ± 8.7## | 123.37 ± 12.96## |
| Nimodipine | 2 | 10 | 11.3 ± 6.6** | 144.77 ± 21.61* |
| Sample A | 200 | 10 | 18.7 ± 8.6** | 151.54 ± 15.90** |
| Sample A | 100 | 10 | 22.9 ± 8.3* | 145.56 ± 27.31* |
| Sample A | 50 | 10 | 27.9 ± 6.0 | 128.11 ± 36.83 |
| Sample B | 200 | 10 | 23.1 ± 9.1* | 143.82 ± 26.85* |
| Sample B | 100 | 10 | 24.7 ± 9.3 | 138.81 ± 27.55 |
| Sample B | 50 | 10 | 29.9 ± 11.5 | 139.75 ± 33.98 |

This invention is not to be limited to the specific embodiments disclosed herein and modifications for various applications and other embodiments are intended to be included within the scope of the appended claims. While this invention has been described in connection with particular examples thereof, the true scope of the invention should not be so limited since other modifications will become apparent to the skilled practitioner upon a study of the drawings, specification, and following claims.

All publications and patent applications mentioned in this specification are indicative of the level of skill of those skilled in the art to which this invention pertains. All publications and patent applications mentioned in this specification are herein incorporated by reference to the same extent as if each individual publication or patent application mentioned in this specification was specifically and individually indicated to be incorporated by reference.

The invention claimed is:

1. A method for the treatment of an ischemic cardiovascular or cerebrovascular disease comprising administrating to a patient in the need thereof a pharmaceutical composition comprising a low molecular weight fucoidan, wherein said low molecular weight fucoidan has been obtained from a sulfated polysaccharide or oligosaccharide substance by a degradation method, and said ischemic cardiovascular and cerebrovascular disease comprises coronary heart disease or stroke.

2. The method of claim 1, wherein said coronary heart disease comprises one or more diseases selected from symptomless coronary heart disease, angina, cardiac infarction, arrhythmia, or sudden death.

3. The method of claim 1, wherein said stroke comprises one or more diseases selected from cerebral hemorrhage or cerebral infarction.

4. The method of claim 1, wherein said low molecular weight fucoidan is from kelp.

5. The method of claim 4, wherein the molecular weight of said low molecular weight fucoidan is between 8000 and 100000 Da.

6. The method of claim 5, wherein the molecular weight of said low molecular weight fucoidan is between 8000 and 60000 Da.

7. The method of claim 6, wherein the molecular weight of said low molecular weight fucoidan is between 8000 and 12000 Da.

8. The method of claim 6, wherein the molecular weight of said low molecular weight fucoidan is between 20000 and 40000 Da.

9. The method of claim 1, wherein said fucoidan is administrated by injection, orally, locally, or intranasally.

10. The method of claim 1, wherein said degradation method comprises acid-catalyzed hydrolysis, base-catalyzed hydrolysis, enzymatic depolymerization, physical degradation, or oxidative depolymerisation.
